(19) United States
(12) Patent Application Publication
(10) Pub. No.: US 2022/0000158 A1
(43) Pub. Date: Jan. 6, 2022

(54) CANNABINOID-CONTAINING CONCENTRATE FOR MAKING A PRODUCT FOR HUMAN CONSUMPTION HAVING AN IMPROVED TASTE PROFILE AND METHODS OF MANUFACTURING SAME

(71) Applicant: HEXO OPERATIONS INC., Ottawa (CA)

(72) Inventors: Max Alsayar, Ottawa (CA); George Elvira, Montreal (CA)

(21) Appl. No.: 17/280,132

(22) PCT Filed: Sep. 26, 2019
(86) PCT No.: PCT/CA2019/051379

§ 371 Date: Mar. 25, 2021

Related U.S. Application Data
(60) Provisional application No. 62/737,036, filed on Sep. 26, 2018.

(51) Int. Cl. A23L 29/10; A23L 2/52; A23L 33/105

(52) U.S. Cl. CPC A23L 29/10; A23L 33/105; A23L 2/52

(57) ABSTRACT

The present disclosure relates to cannabinoid-containing product for human consumption having an improved taste profile and to methods of manufacturing same. The present disclosure also relates to a process for making a cannabinoid-containing product for human consumption, comprising the following steps extraction of a cannabinoid and waxes from cannabis plant material with carbon dioxide under supercritical conditions to obtain an extract containing the cannabinoid and waxes, adding an emulsifier to the extract containing the cannabinoid and waxes to make a cannabinoid-containing emulsion for human consumption.

### CROSS-REFERENCE TO RELATED APPLICATION

The present application claims the benefit of U.S. provisional patent application serial number U.S. 62/737,036 filed on Sep. 26, 2018. The contents of the above-referenced document are incorporated herein by reference in their entirety.

### TECHNICAL FIELD

This application generally relates to the field of cannabinoid-containing products for human consumption having an improved taste profile and to methods of manufacturing same.

### BACKGROUND

Cannabinoids have been used for many years, inter alia, in alleviating pain and inflammatory-related syndromes, spasms, asthma, sleep disorders, depression, loss of appetite and other medical conditions. The cannabinoids are a family of active compounds found mainly in the resin-producing pistillate inflorescences of cannabis plants. Although a variety of cannabinoid compounds have been identified in literature thus far, two compounds in particular have been the main focus of interest for medicinal and recreational uses: tetrahydrocannabinol (THC) and cannabidiol (CBD).

While THC is a psychoactive compound with adverse long-lasting effects on the user, CBD is not regarded as a psychotropic agent and is considered safe for consumption in various routes of administration. Both compounds are typically found as a mixture, at various concentration ranges, in the plant source.

U.S. Pat. No. 7,700,368 describes a process for cannabinoid extraction from plant material using heat decarboxylation to convert cannabinoids in their acid forms to neutral forms (e.g, tetrahydrocannabinolic [THC-A] acid will be decarboxylated to tetrahydrocannabinol [THC]), followed by CO₂ extraction, and then followed by ethanol winterization to remove waxes. Implementing this process results in THC having a chromatographic purity of greater than 99%. The THC is typically a crystalline solid at room temperature.

US 2004/0049059 describes a process for cannabinoid extraction from plant material using CO₂ extraction, followed by ethanol winterization to remove waxes, and followed by decarboxylation to convert cannabinoids in their acid forms to neutral forms. Implementing this process results in pure or nearly pure CBD or THC.

US 2008/0167483 describes a process for cannabinoid extraction from plant material using heat decarboxylation to convert cannabinoids in their acid forms to neutral forms, followed by CO₂ fluid extraction, and followed by ethanol winterization to remove waxes. This document teaches that contrary to expectations, it has determined that cannabinoids are best obtained under subcritical rather than supercritical CO₂ extraction conditions, namely best obtained with a temperature between 8-12° C., and a pressure between 55-65 bar (i.e., 800-950 psi).

A deficiency associated with the above methods, thus, lies in that the extracted cannabinoid extracts often have a bad taste associated therewith, likely due to the presence of residual solvents and/or presence of bitter-tasting molecules or contaminants, which requires the addition of taste masking compounds to finished formulation for use in, for example, edibles. At least some of these deficiencies hinder subsequent formulation and use of cannabinoids in specific applications, such as for example, edibles, pharmaceuticals, beverages, vaping, and the like.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to a process for making a cannabinoid-containing product for human consumption, comprising: extraction of a cannabinoid and waxes from cannabis plant material with carbon dioxide under supercritical conditions to obtain an extract containing the cannabinoid and waxes, adding an emulsifier to the extract containing the cannabinoid and waxes to make a cannabinoid-containing emulsion for human consumption.

As embodied and broadly described herein, the present disclosure also relates to a cannabinoid-containing concentrate for mixing with an emulsifier to make a cannabinoid-containing emulsion for human consumption, the cannabinoid-containing concentrate being free of winterization ethanol and comprising plant waxes.

As embodied and broadly described herein, the present disclosure also relates to an un-distilled cannabinoid-containing concentrate for mixing with an emulsifier to make a cannabinoid-containing emulsion for human consumption, the cannabinoid-containing concentrate being free of winterization ethanol.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A detailed description of specific exemplary embodiments is provided herein below with reference to the accompanying drawings in which:

FIG. 1 is a graph that shows the effect of time of decarboxylation at 120° C. on CBG-A decarboxylation and CBG levels in accordance with a non-limiting embodiment of the present disclosure.

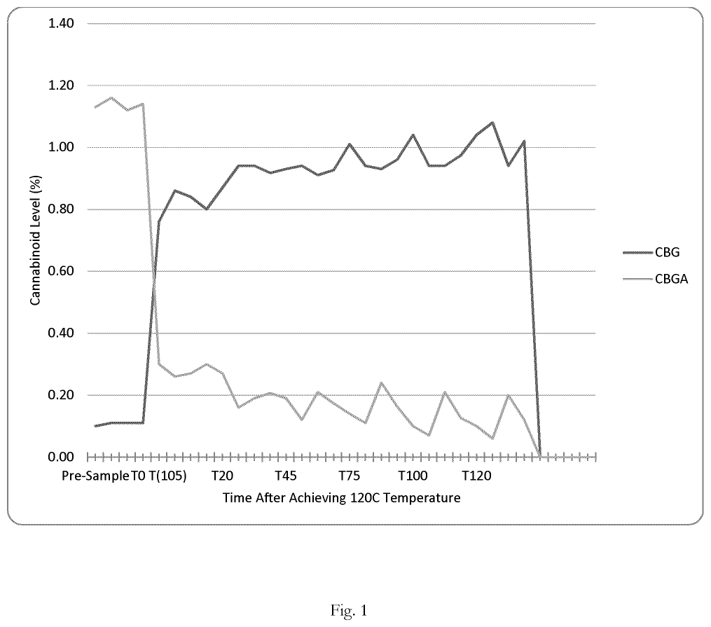

FIG. 2 is a graph that shows the effect of time of decarboxylation at 120° C. on THC-A Decarboxylation, THC and CBN levels.

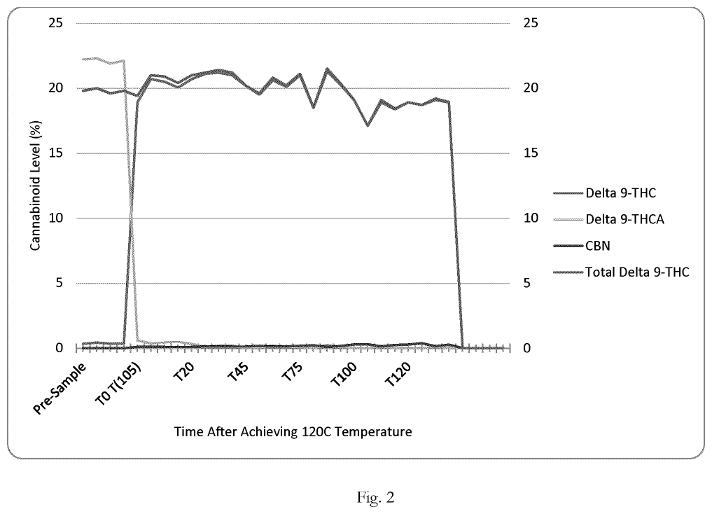

FIG. 3 is a graph that shows the effect of time of decarboxylation at 120° C. on THC-A decarboxylation, THC and CBN Levels in accordance with a non-limiting embodiment of the present disclosure.

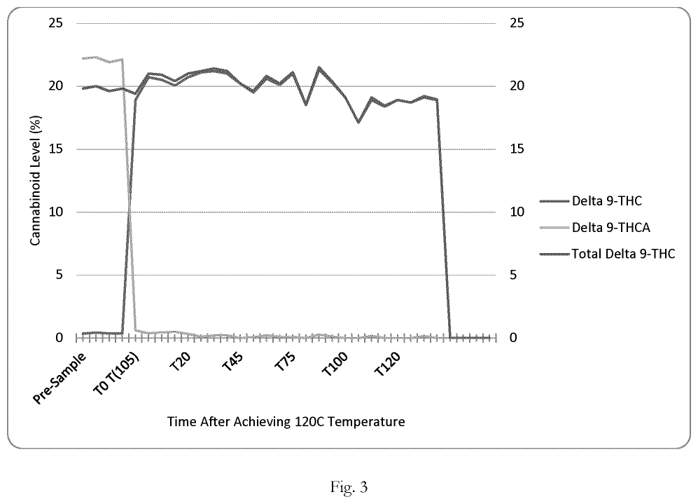

FIG. 4 is a graph that shows the effect of time of decarboxylation at 120° C. on CBG, CBGA, CBN and Total CBG levels in accordance with a non-limiting embodiment of the present disclosure.

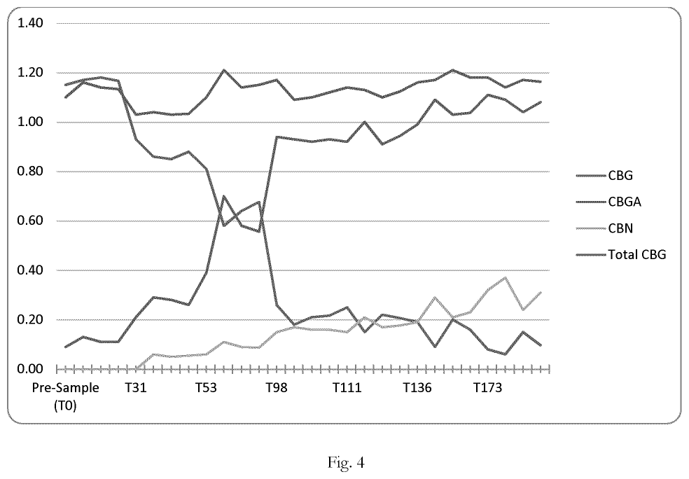

FIG. 5 is a graph that shows the effect of time of decarboxylation at 120° C. on THC-A and THC levels in accordance with a non-limiting embodiment of the present disclosure.

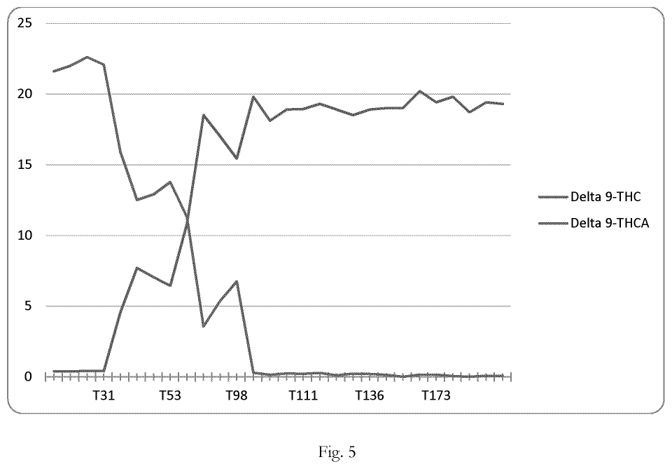

FIG. 6 is a graph that shows the effect of time of decarboxylation at 120° C. on CBG, CBGA, Total CBG and CBN levels in accordance with a non-limiting embodiment of the present disclosure.

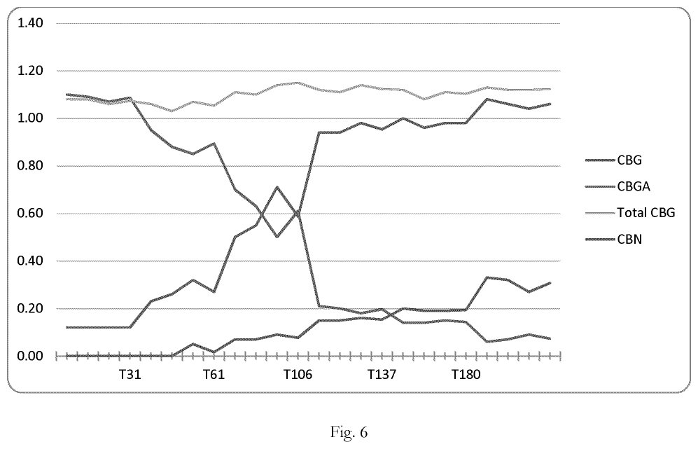

FIG. 7 is a graph that shows the effect of time of decarboxylation at 120° C. on delta 9-THC and delta 9-THCA levels in accordance with a non-limiting embodiment of the present disclosure.

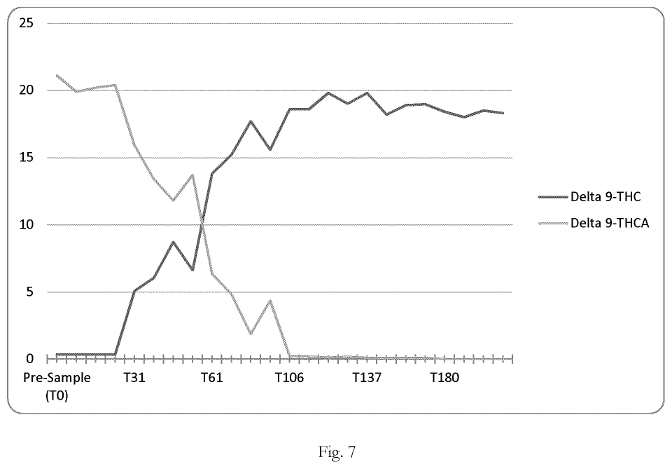

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

The present inventor has surprisingly and unexpectedly discovered a way to manufacture a cannabinoid-containing product for human consumption (such as a beverage) having an improved tasting profile, without requiring addition of taste masking compounds.

Extensive R&D work has been performed to arrive at the herein described extraction process which produces a highly concentrated plant extract material and where the process does not include the conventional winterization step thereby avoiding the use of solvents, such as ethanol or butane. In other words, the extract is free from winterization solvents and is designed to include waxes, have high concentration of a cannabinoid and be for mixing with an emulsifier to make a cannabinoid-containing emulsion for human consumption.

These and other examples of implementation of the present disclosure will become apparent to the person of skill in view of the disclosure as a whole.

### 1. Cannabis

Cannabis is a genus of flowering plants that includes a number of species. The number of species is currently being disputed. There are three different species that have been recognized, namely Cannabis sativa, Cannabis indica and Cannabis ruderalis. Hemp, or industrial hemp, is a strain of the Cannabis sativa plant species that is grown specifically for the industrial uses of its derived products. Hemp has lower concentrations of THC and higher concentrations of cannabidiol (CBD), which decreases or eliminates its psychoactive effects.

The term “Cannabis plant(s)” encompasses wild type Cannabis and also variants thereof, including cannabis chemovars which naturally contain different amounts of the individual cannabinoids. For example, some Cannabis strains have been bred to produce minimal levels of THC, the principal psychoactive constituent responsible for the high associated with it and other strains have been selectively bred to produce high levels of THC and other psychoactive cannabinoids.

Cannabis plants produce a unique family of terpeno-phenolic compounds called cannabinoids, which produce the “high” one experiences from consuming marijuana. There are 483 identifiable chemical constituents known to exist in the cannabis plant, and at least 85 different cannabinoids have been isolated from the plant. The two cannabinoids usually produced in greatest abundance are cannabidiol (CBD) and/or A9-tetrahydrocannabinol (THC), but only THC is psychoactive. Cannabis plants are categorized by their chemical phenotype or “chemotype,” based on the overall amount of THC produced, and on the ratio of THC to CBD. Although overall cannabinoid production is influenced by environmental factors, the THC/CBD ratio is genetically determined and remains fixed throughout the life of a plant. Non-drug plants produce relatively low levels of THC and high levels of CBD, while drug plants produce high levels of THC and low levels of CBD.

### 2. Cannabinoid

A cannabinoid is generally understood to include any chemical compound that acts upon a cannabinoid receptor such as CB1 and CB2. A cannabinoid may include endocannabinoids (produced naturally by humans and animals), phytocannabinoids (found in cannabis and some other plants), and synthetic cannabinoids (manufactured artificially).

Examples of phytocannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ⁹-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ⁸-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CM), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

The terms “cannabidiol” or “CBD” are generally understood to refer to one or more of the following compounds, and, unless a particular other stereoisomer or stereoisomers are specified, includes the compound “Δ²-cannabidiol.” These compounds are: (1) Δ⁵-cannabidiol (2-(6-isopropenyl-3-methyl-5-cyclohexen-1-yl)-5-pentyl-1,3-benzenediol); (2) Δ⁴-cannabidiol (2-(6-isopropenyl-3-methyl-4-cyclohexen-1-yl)-5-pentyl-1,3-benzenediol); (3) Δ³-cannabidiol (2-(6-isopropenyl-3-methyl-3-cyclohexen-1-yl)-5-pentyl-1,3-benzenediol); (4) Δ^{3,7}-cannabidiol (2-(6-isopropenyl-3-methylenecyclohex-1-yl)-5-pentyl-1,3-benzenediol); (5) Δ²-cannabidiol (2-(6-isopropenyl-3-methyl-2-cyclohexen-1-yl)-5-pentyl-1,3-benzenediol); (6) Δ¹-cannabidiol (2-(6-isopropenyl-3-methyl-1-cyclohexen-1-yl)-5-pentyl-1,3-benzenediol); and (7) Δ⁶-cannabidiol (2-(6-isopropenyl-3-methyl-6-cyclohexen-1-yl)-5-pentyl-1,3-benzenediol).

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

In some embodiments, the cannabinoid is a mixture of tetrahydrocannabinol (THC) and cannabidiol (CBD). The w/w ratio of THC to CBD in the liquid formulation may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1.

### 3. Terpene/Terpenoid

A terpene is generally understood to include any organic compound derived biosynthetically from units of isoprene, and the term “terpenoid” generally refers to a chemically modified terpene (e.g., by oxidation). Terpenes are produced by a large variety of plants. As used herein, terpenes include terpenoids. Terpenes may be classified in various ways, such as by their sizes. For example, suitable terpenes may include monoterpenes, sesquiterpenes, or triterpenes. At least some terpenes are expected to interact with, and potentiate the activity of, cannabinoids.

Examples of terpenes known to be extractable from cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof.

Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270, which is incorporated herein by reference in its entirety for all purposes.

### 4. Flavonoid

Flavonoids (or bioflavonoids) (from the Latin word flavus meaning yellow, their color in nature) are a class of plant and fungus secondary metabolites, and can be used as one or more additive in the formulations.

Chemically, flavonoids have the general structure of a 15-carbon skeleton, which consists of two phenyl rings (A and B) and heterocyclic ring (C). This carbon structure can be abbreviated C6-C3-C6. According to the IUPAC nomenclature, they can be classified into: flavonoids or bioflavonoids, isoflavonoids, derived from 3-phenylchromen-4-one (3-phenyl-1,4-benzopyrone) structure, and neoflavonoids, derived from 4-phenylcoumarine (4-phenyl-1,2-benzopyrone) structure.

The three flavonoid classes above are ah ketone-containing compounds, and as such, are anthoxanthins (flavones and flavonols). This class was the first to be termed bioflavonoids. The terms flavonoid and bioflavonoid have also been more loosely used to describe non-ketone polyhydroxy polyphenol compounds, which are more specifically termed flavanoids. The three cycle or heterocycles in the flavonoid backbone are generally called ring A, B and C. Ring A usually shows a phloroglucinol substitution pattern.

Flavonoids are widely distributed in plants, fulfilling many functions. Flavonoids are the most important plant pigments for flower coloration, producing yellow or red/blue pigmentation in petals designed to attract pollinator animals. In higher plants, flavonoids are involved in UV filtration, symbiotic nitrogen fixation and floral pigmentation. They may also act as chemical messengers, physiological regulators, and cell cycle inhibitors. Flavonoids secreted by the root of their host plant help Rhizobia in the infection stage of their symbiotic relationship with legumes like peas, beans, clover, and soy. Rhizobia living in soil can sense the flavonoids and triggers the secretion of Nod factors, which in turn are recognized by the host plant and can lead to root hair deformation and several cellular responses such as ion fluxes and the formation of a root nodule. In addition, some flavonoids have inhibitory activity against organisms that cause plant diseases, e.g. Fusarium oxysporum.

Isoflavones use the 3-phenylchromen-4-one skeleton (with no hydroxyl group substitution on carbon at position 2). Examples include: Genistein, Daidzein, Glycitein, Isoflavanes, Isoflavandiols, Isoflavenes, Coumestans, and Pterocarpans.

Exemplary flavonoids include Apigenin, beta-sitosterol, cannaflavin A, kaempferol, luteolin, orientin, and quercetin.

### 5. Cannabis Oil Extraction

Extraction in natural products chemistry is a separation process comprising the separation of a substance from a matrix of natural materials and includes liquid-liquid extraction, solid phase extraction and what is commonly referred to as super-critical extraction. The distribution of any given compound or composition between two phases is an equilibrium condition described by partition theory. This is based on exactly how the desired material moves from a first solution, typically water or other material capable of dissolving a desired material with a first solubility of the desired material, into second material, typically an organic or other immiscible layer having a second solubility of the desired material layer. Super-critical (supercritical) extraction involves entirely different phenomenon and will be described below.

There exist several types of extraction, including liquid-liquid extraction, solid-phase extraction, solid-phase microextraction, Soxhlet extraction, fizzy extraction and super-critical CO₂ (supercritical carbon dioxide) extraction.

Once various fractions of desired material have been obtained by any method such as any of fractionation and purification methods known in the art, any number of the fractions can be recombined. The recombination can be by simple mixing or by other mechanical means.

### 6. Decarboxylation

THC and CBD are the main medicinally active constituents in Cannabis. However, these constituents are present as the biologically inactive carboxylic acids in Cannabis plants. When extracting THC or CBD from cannabis plants, it has been the practice to convert the storage precursor compounds of THCA and CBDA into their more readily extractable and pharmacologically active forms. THC and CBD acids slowly decarboxylate over time, and applying heat increases the rate of decarboxylation.

Decarboxylation of cannabinoid acids is a function of time and temperature, thus, at higher temperatures a shorter period of time will be taken for complete decarboxylation of a given amount of cannabinoid acid. In selecting appropriate conditions for decarboxylation consideration must, however, be given to minimising thermal degradation of the desirable, pharmacological cannabinoids into undesirable degradation products, particularly thermal degradation of THC to cannabinol (CBN). There is, however, no clear guidance in the art as to what temperatures and time parameters one should use. For example, US 2016/0158298 teaches performing decarboxylation at a temperature that should not exceed 190° F. (about 87° C.), whereas US 2004/0049059 teaches performing decarboxylation at a temperature of 80-140° C., and whereas U.S. Pat. No. 7,700,368 teaches performing decarboxylation in two steps, the first step being at a temperature of 100-110° C. for 10-20 minutes and the second step being at a temperature of 115-125° C. for 45-75 minutes or being at a temperature of 135-145° C. for 15-45 minutes.

There is also no clear guidance as to when one should perform decarboxylation with respect to the supercritical CO₂ extraction and whether inverting the steps has any effect on the end result. For example, U.S. Pat. No. 7,700,368 and US 2006/0167283 teach performing decarboxylation prior to supercritical CO₂ extraction, whereas US 2004/0049059 teaches performing decarboxylation after supercritical CO₂ extraction.

Further, typically, supercritical CO₂ extraction of cannabinoids involves a step of winterization after the CO₂ extraction so as to retain the more polar cannabinoid molecules while ridding the crude extract of most other waxes, which is often referred to as waxy ballast. The secondary extraction or “winterization” is an ethanolic-precipitation for removing waxy ballast and purifying the crude Cannabis extract of wax esters, glycerides, and unsaturated fatty acids, which hinder the extract from a refined liquid state. “Winterization” releases any trapped solvents from the initial extraction from the extremely viscous crude extracts.

The process of removing waxy ballast from crude cannabis extract using “winterization”, involves chilling the crude Cannabis extract to a temperature less than or equal to about 0° C., alternatively less than or equal to below about −10° C., alternatively less than or equal to below about 20° C. for a time period. The time period may be at least 1 hour, alternatively at least about 24 hours, alternatively at least about 48 hours, alternatively at least about 50 hours, alternatively at least about 72 hours. After the chilling freezing period, the crude Cannabis extract can be cold-filtered to remove waxy ballast. For example, a Whatman #1 lab filter with vacuum assist is initially used to remove the material that is insoluble, and secondly the crude extract is run through syringe filters (for example, 0.45 or 0.2 micron filters), which takes out any remaining plant material, as well as any bacteria present.

7. Extraction of Cannabinoid without Winterization

The present inventor has through extensive R&D work surprisingly and unexpectdly discovered a process for producing a cannabis plant extract material containing a high concentration of cannabinoids and having advantageous properties, e.g., for incorporating into an emulsification system for making a product for human consumption and that includes minimal amounts of an emulsifier.

The process for producing such cannabis plant extract containing a high concentration of cannabinoids is exempt of a winterization step. The process may further include a decarboxylation step performed prior to or after a supercritical CO₂ extraction. The cannabis plant extract can then be incorporated into an emulsification system which includes minimal amounts of an emulsifier.

Without being bound by any theory, the present inventor believes that omitting the winterization step in the herein described process is beneficial in that there is no residual winterization solvent (e.g., ethanol, butane, etc.). Alternatively or additionally, omitting the winterization step in the herein described process is beneficial in that waxes remaining in the extract may assist in the emulsification process step such that less emulsifier is required for incorporating the extract into the emulsification system. Ultimately, absence of residual solvent and/or less emulsifier used, translates into a cannabinoid-containing emulsification system that imparts less bitterness to a product containing same, such as for example, an edible, a pharmaceutical oral dosage form, a beverage, etc. In other words, an oral product that incorporates the resulting cannabinoid-containing emulsification system will have an improved taste profile compared to at least some other emulsified cannabinoid containing oral products.

In one practical implementation, the herein described cannabis plant material extract which is ready for incorporating into an emulsification system is, thus, exempt of a winterization solvent, e.g., ethanol. An objective manner to assess whether the extract is exempt of such winterization solvent (e.g., ethanol) is to measure the amount of solvent present in the extract prior to incorporating into the emulsification system. In one embodiment, the herein described extract is thus free from winterization solvent. Another practical way of assessing whether the extract is exempt of such winterization solvent (e.g., ethanol) is to determine whether the extract which is ready for incorporating into an emulsification system still includes plant waxes which are typically undesired and removed through a winterization step. As such, the extract (or concentrate) described herein has a high concentration of a cannabinoid, still includes plant waxes and is ready for mixing with an emulsifier for making a product for human consumption.

The herein described cannabinoid extract can mixed with an emulsifier using a technique and/or an emulsifier as described in any one of 62/725,142, 62/722,422, 62/725,308 and 62/719,926, each of which is herein incorporated by reference herein in its entirety.

In one practical implementation, the cannabinis plant extract can be incorporated into an emsulsifcation system using a ratio of extract to emulsifier which is sufficient to emulsify the concentrated extract such that a cannabinoid-containing product intended for human consumption (e.g., oral administration, such as beverage, edible, pharmaceutical oral dosage form, etc.) contains at least 0.002 mg/ml of cannabinoids and has an improved taste profile compared to at least one other cannabinoid-containing product. For example, such cannabinoid-containing product intended for human consumption may include at least 0.002 mg/ml of a cannabinoid and have a bitterness intensity of 7, or less than 6, or less than 5, or less than 4, based on a quinine sulfate standard solution. The amount of cannabinoids contained in the cannabinoid-containing product intended for human consumption can be up to, for example, 10 mg/ml or more, depending on the particulars of the desired application.

The amount of emulsifier to use will vary depending on the nature of the emulsifier and/or on the emulsifying technique used (e.g., sonication, microwave-assisted, etc.). Nevertheless, the person of skill will readily find the amount to use in order to obtain the herein described cannabinoid-containing product intended for human consumption having an improved taste profile as described herein, provided that the amount selected affords a cannabinoid-containing product intended for human consumption having less than 1.00, or less than 0.90, or less than 0.80, or less than 0.70, or less than 0.60 wt. % emulsifier, for example between 0.04 and 0.65 wt. % emulsifier.

For example, it has been found that using a dilution ratio of the herein described concentrated extract to emulsifier (such as for example, but without being limited to, Tween™-20 or Tween™-80) of, for example but without being limited to, 1:3 to 1:10 produces a cannabinoid-containing emulsification system having advantageous properties. For example, such cannabinoid-containing emulsification system can be incorporated into the herein described cannabinoid-containing product intended for human consumption having a bitterness intensity of 7 based on a quinine sulfate standard solution. Indeed, in a non-limiting practical implementation, incorporating an amount of such cannabinoid-containing emulsification system into a beverage base (for example, 0.1-0.5 g into 150 ml) results in a cannabinoid-containing beverage having between 0.04 and 0.65 wt. % emulsifier and having at least 0.002 mg/ml of cannabinoid.

For the purpose of the present specification, the person of skill will readily understand that a cannabinoid-containing product intended for human consumption may be a beverage, which may include any drink, including water or other liquid; or concentrates, powders, crystals and other mixes or substances which are primarily used to make drinks but are not alone intended to be consumed without adding water or some other liquid.

Advantageously, this bitterness intensity of 7 based on a quinine sulfate standard solution is reachable without having to use any taste masking compounds, such as sugars or sugar substitute which would otherwise be required to mask bitter taste. Advantageously, this may allow one of skill to keep % Brix and/or sucrose equivalent to minimal values such that one may thus adjust the taste profile of the cannabinoid-containing product intended for human consumption, such as a beverage, without necessarily being limited to a sweet taste.

Sugar content of aqueous cannabinoid-containing product intended for human consumption, such as beverages, can be measured as % Brix with a refractormer. Refractive index is a measurement of how light behaves as it passes through the sample. Depending on the sample's composition, light will refract and reflect differently. By measuring this activity with a linear image sensor, the sample's refractive index can be assessed and used to determine its physical properties such as concentration and density. Variations in temperature will affect the density of a solution based on the compound that is present. In digital refractometry the use of temperature compensation is necessary for accurate results. For example, the person of skill can use the HI96801 Digital Refractometer (Hanna Instruments, Inc., RI, USA), which contains a built-in temperature sensor and is programmed with temperature compensation algorithms in accordance with the ICUMSA Methods Book for a percent by weight sucrose solution.

In one non-limiting embodiment, the herein described cannabinoid-containing product intended for oral administration has a Brix value of less than 6, or less than 5, or less than 4, or less than 3, or less than 2° Bx. For reference, a can of Coke™ (Coca Cola Company, USA) has a Brix value of about 9° Bx, which is made of about 85 g sugar in 32 fluid ounces. Carrot juice as well as cranberry juice each has a minimal Brix value of 7.00° Bx, whereas tomato juice has a minimal Brix value of 4.20° Bx, as defined by the standards of the Association of International Juice & Nectar Producers (AIJN), which match or exceed current minimum Brix values as defined by The Fruit Juices and Fruit Nectars (Amendment) Regulations 2011 (USA).

Sweetness is commonly measured by comparison to reference solutions of sucrose. Sucrose is the standard to which all other sweeteners are compared. Humans can recognize sweetness in about 1 or 2% sucrose solution. Coffee is typically sweetened to about the level of 5% sucrose. Soft drinks are usually about as sweet as 10% sucrose. 15% sucrose is really sweet and starts to feel a little syrupy. Taste panelists are often trained to quantitate sweetness on a 15 cm line scale, for convenience, using 2-15% sucrose solutions as references. Other sweeteners are then tasted at a series of dilutions to determine the concentration that is as sweet as a given percent sucrose reference. For example, if a 1% solution of sweetener X is as sweet as a 10% sucrose solution, then sweetener X is said to be 10 times as potent as sucrose.

For a detailed discussion, see “A Systematic Study of Concentration-Response Relationships of Sweeteners,” G. E. DuBois, D. E. Walters, S. S. Schiffman, Z. S. Warwick, B. J. Booth, S. D. Pecore, K. Gibes, B. T. Carr, and L. M. Brands, in Sweeteners: Discovery, Molecular Design and Chemoreception, D. E. Walters, F. T. Orthoefer, and G. E. DuBois, Eds., American Chemical Society, Washington, D.C. (1991), pp 261-276.

In one non-limiting embodiment, the herein described cannabinoid-containing product intended for oral administration has a sweetness value comparable to a level of less than 5%, or less than 4%, or less than 3%, or less than 2%, or less than 1% sucrose.

The bitterness intensity based on the quinine sulfate standard solution as used in the present specification refers to, in an sensory test based on 10 standard solutions each adjusted in advance to have different levels of bitterness intensity which differ by equal increments using quinine sulfate (refer to Table 1 of Example, Indow, T, Perception & Psychophysics, Vol. 5 (1969), pp. 347 to 351), the bitterness intensity of the quinine sulfate standard solution which was recognized by a subject, among those standard solutions, to have an equal bitterness intensity to the test cannabinoid-containing product intended for oral administration. More specifically, the bitterness intensity is determined by the following procedure. Firstly, five healthy people having a normal sense of taste are assigned to be subjects, and each subject holds each quinine sulfate standard solution in the mouth in ascending order of concentration to memorize the bitterness intensity. Subsequently, each subject holds a test cannabinoid-containing product intended for oral administration in the mouth to recognize the degree of bitterness, and from among the quinine sulfate standard solutions, determines one having the closest bitterness level. Then, the values of bitterness intensity determined by each subject are averaged out and provided as the bitterness intensity of the test cannabinoid-containing product intended for oral administration. It is noted that the smaller the bitterness intensity, the weaker the bitterness.

In one non-limiting practical implementation, there is thus provided a process for reducing bitterness in a cannabinoid-containing product intended for oral administration (e.g., edible, beverage, pharmaceutical oral dosage, nutraceutical, and the like). This process includes extraction of cannabis plant material with carbon dioxide under supercritical conditions to obtain a plant extract containing at least one cannabinoid. A step of decarboxylation, for activating the at least one cannabinoid, can be performed prior to or after the step of carbon dioxide extraction. Then, incorporating the extract into an emulsification system by adding a minimal amount of an emulsifier to the extract to obtain a cannabinoid-containing emulsification system. Then, incorporating an amount of the cannabinoid-containing emulsification system into a product intended for oral administration such that the product includes at least 0.002 mg/ml of the at least one cannabinoid, and less than 1.00 wt. % of the emulsifier. Advantageously, this process does not require a winterization step of the plant extract.

The product intended for oral administration can be any product such as those described in any one of 62/725,142, 62/722,422, 62/725,308 and 62/719,926, each of which is herein incorporated by reference herein in its entirety, and which include at least edibles, beverages, and the like.

It was observed that extracted cannabis resin contained a large amount of cannabinoids in their acid forms. Cannabinoids in their acid forms like THC-A have a delayed onset of therapeutic affect when compared to the neutral THC. A method was developed for carrying out decarboxylation of the cannabinoids from their acid forms to the neutral forms. The method was applied to milled cannabis. The cannabis was milled based on a milling method.

The decarboxylation method was done by applying heat uniformly to milled cannabis material, in a calibrated scientific oven. The work was conducted on the target temperature needed and the time of exposure to complete the reaction. The work for defining the extraction parameters started with a DOE. The investigated temperature range was 110° C. to 130° C. and the exposure time (residence time) was evaluated from 1-2 hours. There were very few scientific sources that studied this reaction at the time, and no predictive model existed for such a large scale application.

The experimental work was initially done over small batches of 1-2 kg of milled cannabis. A temperature probe was used to measure the temperature of the cannabis material, and this was contrasted against the ambient temperature of the oven. This method was repeated over larges batches of 5 kg with the same variation of temperatures. After completing each cycle, samples of cannabis were taken at the beginning, various mid-process points, and finally at the end. This work attempted to model the reaction of converting cannabinoids in their acid forms to neutral, in an effort to develop new knowledge surrounding the rate of reaction for THC-A and CBD-A.

TABLE 0

| | time | Wt. | [CBD] | [CBDA] | [CBN] | Temp |
|---|---|---|---|---|---|---|
| T 154C | (min) | (g) | % | % | % | (° C.) |
| Test_−01 | −60 | 2 | 14.7 | 0.25 | ND | 120 |
| Test_00 | 0 | 2 | 14.2 | 0.11 | ND | 120 |
| Test_01 | 5 | 2 | 13.5 | 0.08 | <0.05 | 135 |
| Test_02 | 15 | 2 | 14.0 | 0.07 | 0.05 | 145 |
| Test_03 | 20 | 2 | 13.6 | <0.05 | 0.07 | 150 |
| Test_04 | 25 | 2 | 13.3 | <0.05 | 0.08 | 150 |
| Test_05 | 30 | 2 | 13.6 | <0.05 | 0.09 | 150 |

TABLE 0.5

| Temp | time | [CBD] | [CBDA] | [CBN] |
|---|---|---|---|---|
| (° C.) | (min) | % | % | % |
| 135 | +5 | 13.5 | 0.08 | <0.05 |
| 145 | +15 | 14 | 0.07 | 0.05 |
| 150 | +20 | 13.6 | <0.05 | 0.07 |
| 150 | +25 | 13.3 | <0.05 | 0.08 |
| 150 | +30 | 13.6 | <0.05 | 0.09 |

### EXAMPLES

The following examples describe some exemplary modes of making and practicing certain compositions that are described herein. It should be understood that these examples are for illustrative purposes only and are not meant to limit the scope of the compositions and methods described herein.

### Example 1

In this example, compositions containing an emulsion having particle sizes >1000 nm (Formulation 1), 200 nm (Formulation 2) and 40 nm (Formulation 3) were made.

Cannabinoid based emulsions having a particle size of 40 nm and 200 nm are provided below in Tables 1 and 2. Cannabinoid based emulsions having a particle size of >1000 nm were prepared based on the formulae set out in Tables 1 and 2, without the additional sonication step. These exemplary formulations span the range from nano-emulsions to macro-emulsions. The foregoing emulsions were prepared as follows:
1. The water and oil phase ingredients were solubilized separately using heat and stirring. In particular, the water phase is comprised of water, Tween™ 80, ascorbic acid and EDTA and mixed at 60° C. with a magnetic stir bar for 30 minutes. The oil phase is comprised of Labrafac™ lipophile WL 1349, Tocobiol™, lecithin and THC distillate and mixed at 60° C. with a magnetic stir bar for 30 minutes.
2. Once the respective water and oil phases have been prepared they were combined while mixing with a high shear homogenizer at 8000-10000 rpm. The oil phase was added slowly to the water phase over 5 minutes and once completely the resultant emulsion was mixed for an additional 15 minutes. The resultant mixture is a macro-emulsion with a particle size >1000 nm.
3. To generate the 40 nm and 200 nm nano-emulsions, high energy sonication was applied to the macro-emulsions for 10 minutes with 100% amplitude using an LSP-500 Ultrasonic Processor (Sonomechanics, Florida, USA).

Using the same excipient components and tuning the ratio of emulsifiers to achieve the different particle sizes eliminates the experimental uncertainty in permeation data (see in later example) interpretation that would normally be associated if using different emulsifier combinations to achieve the different particle sizes.

Particle size of all nanoemulsions was measured in water solution at 25° C. using dynamic light scattering (DLS). All samples in the present disclosure have been analyzed at a dilution of 1/20 in purified water using a LiteSizer™ (Anton Paar GmbH, Germany).

TABLE 1

| Excipients | Mass (g) | % Blend |
|---|---|---|
| THC Distillate-03 | 18.75 | 2.5 |
| Labrafac lipophile | 20 | 2.67 |
| Ascorbic acid | 4.5 | 0.6 |
| Tocobiol | 3.75 | 0.5 |
| EDTA | 0.1 | 0.01 |
| Lecithin | 15 | 2 |
| Tween 80 | 60 | 8 |
| Water | 627.9 | 83.72 |

TABLE 2

| Excipients | Mass (g) | % Blend |
|---|---|---|
| THC Distillate-03 | 18.75 | 2.5 |
| Labrafac lipophile | 20 | 2.67 |
| Ascorbic acid | 3.75 | 0.5 |
| Tocobiol | 4.5 | 0.6 |
| EDTA | 0.1 | 0.01 |
| Lecithin | 10 | 1.33 |
| Tween80 | 15 | 2 |
| Water | 677.9 | 90.39 |

The results clearly demonstrate that the approach of the present disclosure allows for making an emulsion using less emulsifiers than in known procedures.

### Example 2

In this example, a composition containing THC with a particle size <100 nm was made.

1,000 mg of THC-containing cannabis oil was mixed with 50 mg of poly(ethylene glycol) monooleate with an appropriate amount of ethanol in a container to obtain an oil phase mixture. The oil phase mixture was heated at 50° C. until a liquid oil phase was obtained. In a separate container, 50 mg of sodium oleate were dissolved into 20 mL of deionized water to form an aqueous phase mixture. The oil phase mixture was added to the aqueous phase mixture and the combined mixture was mixed with a high shear mixer to obtain a coarse emulsion. A T25 (IKA, Staufen, Germany) at 8,000 rpm for 5 minutes can be used here. The coarse emulsion was mixed with a microfluidizer to further homogenize the emulsion and obtain the first microencapsulation composition containing THC with a particle size <100 nm. A Nano DeBEE, (Westwood, Mass., USA) at 20,000 psi for 8-12 cycles can be used here.

### Example 3

In this example, a composition containing CBD with a PSD of ≥200 nm was made.

5 g of limonene and 25 g of whey protein isolate were mixed with 70 g of water by stirring. The mixture was left for 24 hours to allow complete biopolymer hydration and saturation. After 24 hours, the mixture was homogenized using a sonicator. A Digital Sonifier 450 (Branson Ultrasonic Corporation, USA) at 160 W for 2 minutes can be used here. After homogenization, the emulsion was placed in an ice bath until the emulsion reached room temperature so as to obtain the second microencapsulation composition containing CBD with a PSD of ≥200 nm.

### Example 4

In this example, a composition containing CBD with a PSD of ≥200 nm was made.

5 g of CBD-containing cannabis oil extract was mixed with 0.794 g Tween 80, 4.206 g Span 80, and 90 g distilled water in a test tube. The resulting mixture was heated to 70° C. and immediately homogenized to obtain the second microencapsulation composition containing CBD with a PSD of ≥200 nm. An Ultra Turrax T 25 device (IKA, Staufen, Germany) at 13,400 rpm for 15 minutes can be used here.

### Example 5

In this example, a composition containing CBD with a PSD of ≥200 nm was made.

0.794 g Tween 80 was dissolved in 90 g distilled water to form an aqueous phase. 4.206 g Span 80 was dissolved in 5 g CBD cannabis oil to form an oil phase. Both the aqueous and oil phases were heated to 70° C. and maintained at this temperature. The aqueous phase was added drop-wise to the oil phase, while stirring the oil phase to obtain the second microencapsulation composition containing CBD with a PSD of ≥200 nm. An RZR Heidolph homogenizer (Heidolph Instruments GmbH & Co. KG, Schwabach, Germany) at 1050 rpm over a duration of 30 min can be used here.

### Example 6

In this example, a composition containing CBD with a PSD of ≥200 nm was made.

The same procedure as described in Example 5 was repeated except that 1.262 g Tween 80 was dissolved in 90 g distilled water to form the aqueous phase and 3.738 g Span 80 was dissolved in 5 g CBD cannabis oil extract to form the oil phase.

### Example 7

In this example, a composition containing CBD with a PSD of ≥200 nm was made.

The same procedure as described in Example 5 was repeated except that 1.729 g Tween 80 was dissolved in 90 g distilled water to form the aqueous phase and 3.271 g Span 80 was dissolved in 5 g CBD cannabis oil extract to form the oil phase.

### Example 8

In this example, a composition containing CBD with a PSD of ≥200 nm was made.

The same procedure as described in Example 5 was repeated except that 2.196 g Tween 80 was dissolved in 90 g distilled water to form the aqueous phase and 2.804 g Span 80 was dissolved in 5 g CBD cannabis oil extract to form the oil phase.

### Example 9

In this example, a composition containing CBD with a PSD of ≥200 nm was made.

The same procedure as described in Example 5 was repeated except that 2.664 g Tween 80 was dissolved in 90 g distilled water to form the aqueous phase and 2.336 g Span 80 was dissolved in 5 g CBD cannabis oil extract to form the oil phase.

### Example 10

In this example, a composition containing CBD with a PSD of ≥200 nm was made.

The same procedure as described in Example 5 was repeated except that 2.826 g Tween 80 was dissolved in 90 g distilled water to form the aqueous phase and 2.174 g Span 80 was dissolved in 5 g CBD cannabis oil extract to form the oil phase.

### Example 11

In this example, a composition containing CBD with a PSD of ≥200 nm was made.

The same procedure as described in Example 5 was repeated except that 3.370 g Tween 80 was dissolved in 90 g distilled water to form the aqueous phase and 1.630 g Span 80 was dissolved in 5 g CBD cannabis oil extract to form the oil phase.

### Example 12

In this example, a composition containing CBD with a PSD of ≥200 nm was made.

The same procedure as described in Example 5 was repeated except that 3.913 g Tween 80 was dissolved in 90 g distilled water to form the aqueous phase and 1.087 g Span 80 was dissolved in 5 g CBD cannabis oil extract to form the oil phase.

### Example 13

In this example, various compositions containing THC at 2.5 wt. % were made in accordance with embodiments of the present disclosure and as per the procedure set forth in Example 1.

TABLE 3

| Ingredient | Mass (g) | % Blend |
|---|---|---|
| THC Distillate | 3.75 | 2.50 |
| Coconut Oil | 4 | 2.67 |
| Lecithin sunflower | 3 | 2 |
| Tween 80 | 12 | 8 |
| Water | 127.25 | 84.83 |

| | |
|---|---|
| PSD | 59.4 nm |

TABLE 4

| Ingredients | Mass (g) | % Blend |
|---|---|---|
| THC Distillate | 3.75 | 2.50 |
| Coconut Oil | 4.00 | 2.67 |
| Span 80 | 3.00 | 2.00 |
| Tween 80 | 12.00 | 8.00 |
| Water | 127.25 | 84.83 |

| | |
|---|---|
| PSD | 122.7 nm |

TABLE 5

| Ingredients | Mass (g) | % Blend |
|---|---|---|
| THC Distillate | 3.75 | 2.50 |
| Coconut Oil | 4.00 | 2.67 |
| Brij ™ C2-SO | 1.50 | 1.00 |
| Tween 80 | 11.00 | 7.33 |
| Water (g): | 129.75 | 86.50 |

| | |
|---|---|
| PSD | 87.4 nm |

TABLE 6

| Ingredients | Mass (g) | % Blend |
|---|---|---|
| THC Distillate | 3.75 | 2.50 |
| Coconut Oil | 4.00 | 2.67 |
| Vit E TPGS | 3.00 | 2.00 |
| Tween 80 | 9.00 | 6.00 |
| Lecithin sunflower | 3.00 | 2.00 |
| Water | 127.25 | 84.83 |

| | |
|---|---|
| PSD | 36 nm |

TABLE 7

| Ingredients | Mass (g) | % Blend |
|---|---|---|
| THC Distillate | 3.75 | 1.74 |
| Vit E TPGS | 3.75 | 1.74 |
| Ethanol | 8.00 | 3.71 |
| Tween 20 | 150.00 | 69.61 |
| Water | 50.00 | 23.20 |

| | |
|---|---|
| PSD | 10 nm |

### Example 14—Precursor Composition

In this example, a precursor composition in accordance with an embodiment of the present disclosure was made by gently mixing a composition containing THC with a particle size <100 nm (as described in any one of the previous examples) and a composition containing CBD with a particle size >200 nm (as described in any one of the previous examples).

The compositions were gently mixed to obtain a precursor composition in accordance with an embodiment of the present disclosure.

### Example 15

A THC precursor composition obtained as per the procedure set out in Example 16 was incorporated into a beverage base to obtain a cannabis-infused beverage which was canned into a packaging unit container (e.g., 355 ml can) so as to include 10 mg THC and 100 mg CBD per container in accordance with an embodiment of the present disclosure.

### Example 16

Beverages were obtained by blending a precursor composition into a beverage base. The emulsion was obtained using polysorbates (Tween-20) and Tween-80 to emulsify crude cannabis resin (CBD-resin) into a selection of 12 beverage bases.

Different mixing methods were used and tested:
1. Mix the resin and Tween-20 or Tween-80 at room temperature
2. Mix the resin and Tween-20 or Tween-80 at room temperature, and sonicate the mixture
3. Mix the resin and Tween-20 or Tween-80 in water bath at 35° C., and sonicate the mixture.

Ratios of resin to surfactant of 1:3, 1:5 and 1:10 resulted in a homogenised mixture. The mixture of the surfactant and the resin was added to a beverage base by sonication with the exception of manual mixing for a carbonated beverage. Lab results confirm successful emulsification.

Cyclodextrin and the resin in a weight to weight ratio of 1:1 was manually mixed. The amount of cyclodextrin was increased until cyclodextrin was able to absorb all of the cannabis resin into the powder. This powder was then mixed with a base beverage using the same methods.

Lab results confirm successful emulsification, and the cannabis powder contained 11.4 wt. % THC.

The above beverages were processed using a fining agent under fining conditions to improve the clarity of the beverages, e.g., to obtain a turbidity of less than 0.05 cm⁻¹ at 600 nm.

Several fining agents and fining conditions were used. In an illustrative example, gelatin was used at various concentrations (wt./wt. %) from 0.7 wt. % up to 120 wt. %. The beverages were stored at 4° C. for at least 3 days, then processed with gelatin, and returned to storage at 4° C. for another 4 days.

There was heavy settlement in the beverages demonstrating that the fining procedure was complete. The clearest of the set was decanted. The lab results confirm that processing cannabinoid containing beverages using a fining agent under fining conditions improved the turbidity of the beverages so as to obtain a turbidity of less than 0.05 cm⁻¹ at 600 nm.

For example, it was observed that preferably one should use a concentration of 2% (wt./wt.) of gelatin in the cannabinoid-containing beverage so as to minimize settlement of the cannabinoid and emulsifying agent. For example, using 0.8 wt. %-1.0 wt. % gelatin produced a clear solution without affecting the cannabinoids content.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Although the foregoing invention has been described in some detail by way of illustration and example for purposes of clarity of understanding, it is readily apparent to those of ordinary skill in the art in light of the teachings of this invention that certain changes and modifications may be made thereto without departing from the scope of the appended claims.

It is to be understood that any numerical value inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

1.-33. (canceled)

34. A process for making a cannabinoid-containing product for human consumption, comprising the following steps:
a) extracting a cannabinoid and waxes from cannabis plant material with carbon dioxide under supercritical conditions to obtain an extract containing the cannabinoid and waxes, and
b) adding an emulsifier to the extract containing the cannabinoid and waxes to make a cannabinoid-containing emulsion for human consumption.

35. The process of claim 34, wherein the process is free of a step to remove the waxes.

36. The process of claim 34, comprising performing a decarboxylation step prior to step (a) so as to obtain an activated cannabinoid.

37. The process of claim 34, comprising performing a decarboxylation step after step (a) and before step (b) so as to obtain an activated cannabinoid.

38. The process of claim 34, comprising incorporating the cannabinoid-containing emulsion for human consumption into a beverage.

39. The process of claim 38, wherein the beverage includes at least 0.002 mg/ml of the cannabinoid.

40. The process of claim 34, wherein step (b) includes adding the emulsifier in a ratio of emulsifier to extract of 1:3 to 1:10.

41. The process of claim 34, wherein the emulsifier is a polysaccharide-based emulsifier, a protein-based emulsifier, a small-molecule surfactant, or a mixture thereof.

42. The process of claim 34, wherein the cannabinoid includes one or both of tetrahydrocannabinol (THC) and cannabidiol (CBD).

43. An un-distilled cannabinoid-containing concentrate for mixing with an emulsifier to make a cannabinoid-containing emulsion for human consumption, the cannabinoid-containing concentrate being free of winterization ethanol.

44. The un-distilled cannabinoid-containing concentrate of claim 43, wherein the emulsifier is one of a polysaccharide-based emulsifier, a protein-based emulsifier, a small-molecule surfactant, or a mixture thereof.

45. The un-distilled cannabinoid-containing concentrate of claim 43, wherein the cannabinoid includes one or both of tetrahydrocannabinol (THC) and cannabidiol (CBD).

46. The un-distilled cannabinoid-containing concentrate of claim 43, comprising incorporating the cannabinoid-containing emulsion for human consumption into a beverage.

47. The un-distilled cannabinoid-containing concentrate of claim 46, wherein the beverage includes at least 0.002 mg/ml of the cannabinoid.

48. A cannabinoid-containing concentrate for mixing with an emulsifier to make a cannabinoid-containing emulsion for human consumption, the cannabinoid-containing concentrate being free of winterization ethanol and comprising plant waxes.

49. The cannabinoid-containing concentrate of claim 48, wherein the emulsifier is selected from a polysaccharide-based emulsifier, a protein-based emulsifier, a small-molecule surfactant, or a mixture thereof.

50. The cannabinoid-containing concentrate of claim 48, wherein the cannabinoid includes one or both of tetrahydrocannabinol (THC) and cannabidiol (CBD).

51. The cannabinoid-containing concentrate of claim 48, comprising incorporating the cannabinoid-containing emulsion for human consumption into a beverage.

52. The cannabinoid-containing concentrate of claim 51, wherein the beverage includes at least 0.002 mg/ml of the cannabinoid.
